(19) United States
(12) Patent Application Publication
(10) Pub. No.: US 2024/0300893 A1
(43) Pub. Date: Sep. 12, 2024

(54) SMALL MOLECULE COMPOUNDS TARGETING SRSF6 PROTEIN AND THEIR PREPARATION METHODS AND APPLICATIONS

(71) Applicant: CHINA PHARMACEUTICAL UNIVERSITY, Jiangsu (CN)

(72) Inventors: Wenying YU, Jiangsu (CN); Maode LAI, Jiangsu (CN); Tong WANG, Jiangsu (CN); Cong LI, Jiangsu (CN); Honghe ZHANG, Jiangsu (CN); Zhiyu LI, Jiangsu (CN)

(73) Assignee: CHINA PHARMACEUTICAL UNIVERSITY, Jiangsu (CN)

(21) Appl. No.: 18/652,809

(22) Filed: May 1, 2024

Related U.S. Application Data
(63) Continuation of application No. PCT/CN2022/072519, filed on Jan. 18, 2022.

(30) Foreign Application Priority Data
Nov. 3, 2021 (CN) 202111293452.X

(51) Int. Cl. C07D 211/86; A61K 31/4704

(52) U.S. Cl. CPC C07D 211/86; A61K 31/4704

(57) ABSTRACT

The invention discloses small molecular compounds targeting SRSF6 protein, its preparation method and applications. The compound is shown in formula (I). This molecule can selectively inhibit abnormal cells with high expression of SRSF6, thereby reducing the occurrence of abnormal alternative splicing events and inhibiting the occurrence and development of tumors, especially colorectal tumors. It can be used as a candidate new drug for anti-colorectal cancer.

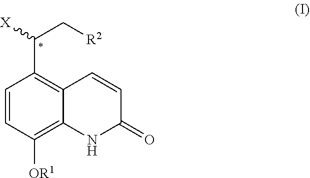

### CROSS-REFERENCE TO RELATED APPLICATION

The present application is a continuation of PCT application No. PCT/CN2022/072519, filed on Jan. 18, 2022, which claims the priority of China Patent Application No. 202111293452.X, filed on Nov. 3, 2021. The entirety of each of the above-mentioned patent applications is incorporated by reference herein and made a part of this specification.

### BACKGROUND

### Technical Field

The present invention relates to the field of medicinal chemistry and pharmacotherapy, in particular to a small molecule compound targeting SRSF6 protein and a preparation method and use thereof.

### Description of Related Art

Aberrant alternative splicing (AS) is common in the occurrence and development of tumors, and most tumor-related genes have abnormal physiological functions due to aberrant AS. SR (serine/arginine-rich) proteins are a class of serine/arginine-rich splicing factors. There are 12 SRSF members in the protein family, and their common structure includes an N-terminal RNA recognition motif, RRM domain) and the C-terminal arginine/serine-rich domain (RS domain). SR proteins bind specifically to pre-mRNA to determine the splicing site. The RS domain recruits other splicing factors to participate in RNA splicing. The dysregulated expression of SR proteins can lead to abnormalities in various splicing processes, and then promote the occurrence of malignant tumors. Serine/arginine-rich splicing factor 6 (SRSF6) protein is a member of the SR protein family. Studies have shown that its expression is up-regulated in colorectal cancer, lung cancer and other cancers. SRSF6 can bind to the motif of exon23 of cell adhesion factor ZO-1 and activate the splicing of exon23 of ZO-1, leading to the occurrence of abnormal alternative splicing, and then promote the occurrence and development of colorectal cancer.

Colorectal cancer (CRC) is a common malignant tumor of the digestive tract formed by the inner wall of the colon or rectum. It is one of the malignant tumors that pose a great threat to human survival and health in the world today, and ranks the third in the death spectrum of cancer in western developed countries. With the change of lifestyle and dietary structure of residents, the incidence of colorectal cancer in China has shown a significant upward trend, and it has ranked the second among new cancers in China. Colorectal cancer has a complex etiology and can occur in any part of the colon or rectum. Indacaterol is used in adults with chronic obstructive pulmonary disease (COPD), but studies have shown that it can be used in the treatment of tumors, especially colorectal tumors; It can inhibit cell proliferation, migration and invasion by regulating alternative splicing in cells.

### SUMMARY

The present invention provides a small molecular compound targeting SRSF6 protein and a preparation method and use thereof. This molecule can selectively inhibit abnormal cells with high expression of SRSF6, thereby reducing the occurrence of abnormal alternative splicing events and inhibiting the occurrence and development of tumors, especially colorectal tumors. It can be used as a candidate new drug for anti-colorectal cancer.

The present invention provides a compound as shown in the general formula (I):

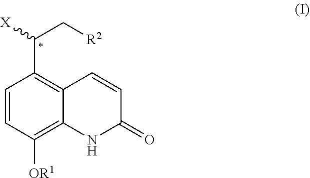

where R¹ is selected from a substituted or unsubstituted C1-C3 linear alkyl group, a substituted or unsubstituted C1-C3 alkenyl group, a substituted or unsubstituted C6-C10 aryl group, a substituted or unsubstituted C6-C10 aralkyl group, a C6-C10 haloalkyl group, or a C6-C10 heteroaryl group, wherein the heteroatoms of the heteroaryl group are selected independently from N, O, or S;
R² is selected from a C1-C3 alkoxy group, substituted amino group, C1-C8 aliphatic amine group, saturated or unsaturated substituted or unsubstituted C6-C10 aryl group, substituted or unsubstituted C6-C10 heteroaryl group, halogenated aromatic carbocyclic amine group or substituted or unsubstituted 5- to 7-membered heterocyclic group, wherein, The heteroatoms in the heteroaryl group or heterocyclic group are independently selected from N, O, or S, and the halogen atoms refer to fluorine, chlorine, bromine, or iodine; The substituted amino group is replaced by one to three of the following substituents: C1-C4 alkyl, 5, 6-diethyl indenyl, substituted or unsubstituted phenyl, indenyl, indolyl, methylsulfonyl substituted piperidinyl, benzothiazolyl, benzimidazolyl, respectively;
X is selected from a hydroxyl group or a halogen, and the halogen is selected from fluorine, chlorine, bromine and iodine;
The chiral center * is the S or the R configuration.

Preferably, R¹ is chosen from a methyl group, an allyl group, a substituted or unsubstituted benzyl group, a substituted or unsubstituted pyridyl group or a triazole; The substituted or unsubstituted benzyl group is independently substituted by one to three of the following groups: a halogen or a C1-C3 alkyl group;

R² selected from R² selected from methoxy, 5,6-diethyl-2,3-dihydro-1H-indin-2-amine, 2-aminoindenyl, aniline, bromoaniline, heteroarylamine, C1-C4 aliphatic amine, morpholinyl, piperidinyl, pyrrolidine, substituted or unsubstituted piperazinyl, substituted or unsubstituted hyperpiperazinyl, indolyl, 2,3-dihydroindenyl, Wherein a substituted or unsubstituted piperazinyl group or a substituted or unsubstituted hyperpiperazinyl group is replaced by one to three independent substituents: C1-C4 alkyl group, substituted or unsubstituted phenyl group, or substituted or unsubstituted benzyl group, wherein the phenyl or benzyl group is substituted by a halogen or C1-C3 alkyl group.

Preferably, R¹ is selected from a benzyl group, a 3-substituted benzyl group, a 4-substituted benzyl group or a multi-substituted benzyl group, the substituent of which is a methyl group or a halogen;

R² is selected from 5,6-diethyl-2,3-dihydro-1H-indenin-2-amino, N-phenylpiperazinyl, N-phenylhomopiperazinyl, bromoaniline, 2,3-dihydroindolyl or 2,3-dihydroindenyl; The N-phenylpiperazinyl is selected from: benzyl substituted piperazinyl, methyl benzyl substituted piperazinyl or benzyl bromide substituted piperazinyl; N-benzyl hyperpiperazinyl is selected from: benzyl substituted for hyperpiperazinyl, methyl benzyl substituted for hyperpiperazinyl, or benzyl bromide substituted for hyperpiperazinyl.

Preferably, the polysubstituted benzyl group is a 3,5-position halobenzyl group, a 3,4-halobenzyl group or a halobenzyl group;

Preferably, the R¹ is selected from benzyl, 4-methylbenzyl, 3-fluorobenzyl, 4-fluorobenzyl, 3,5-difluorobenzyl, 3,4-difluorobenzyl, perfluorobenzyl;

R² was selected from 5,6-diethyl-2,3-dihydro-1H-indenin-2-amine, 2-bromoaniline, 2,3-dihydroindolyl, 2,3-dihydroindenyl, benzyl-substituted piperazinyl, 4-methylbenzyl-substituted piperazinyl, 4-bromobenzyl-substituted piperazinyl, benzyl-substituted high-piperazinyl, 4-methylbenzyl-substituted high-piperazinyl.

In some embodiments, the compound is preferably from the following compounds:

| | |
|---|---|
| (S)-5-(2-((5,6-diethyl-2,3-dihydro-1H-inden-2-yl)amino)- 1-hydroxyethyl)-8-((4-fluorobenzyl)oxy)quinolin-2(1H)- one (I₁/LYW2) | 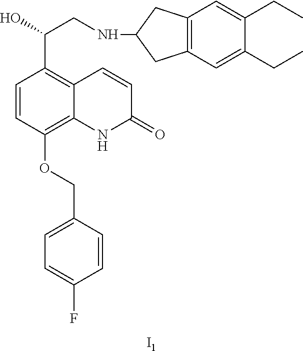 [SMILES: CCc1cc2c(cc1CC)CC(NC[C@@H](O)c1ccc(OCc3ccc(F)cc3)c3[nH]c(=O)ccc13)C2 ; "I1"] |
| (R)-5-(2-((5,6-diethyl-2,3-dihydro-1H-inden-2- yl)amino)-1-hydroxyethyl)-8-((4- fluorobenzyl)oxy)quinolin-2(1H)-one (I₂) | 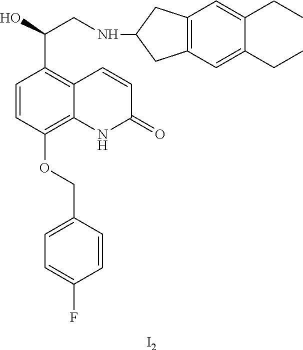 [SMILES: CCc1cc2c(cc1CC)CC(NC[C@H](O)c1ccc(OCc3ccc(F)cc3)c3[nH]c(=O)ccc13)C2 ; "I2"] |
| (R)-5-(2-((5,6-diethyl-2,3-dihydro-1H-inden-2- yl)amino)-1-hydroxyethyl)-8-((4- fluorobenzyl)oxy)quinolin-2(1H)-one (I₃) | 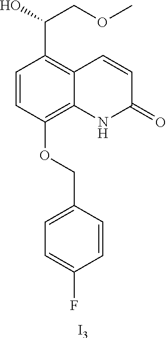 |
| (S)-5-(2-((5,6-diethyl-2,3-dihydro-1H-inden-2-yl)amino)- 1-hydroxyethyl)-8-((4-methylbenzyl)oxy)quinolin-2(1H)- one (I₄) | 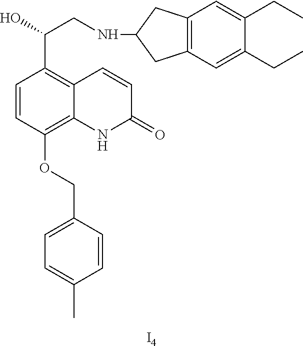 [SMILES: CCc1cc2c(cc1CC)CC(NC[C@@H](O)c1ccc(OCc3ccc(C)cc3)c3[nH]c(=O)ccc13)C2 ; "I4"] |
| (R)-5-(2-((5,6-diethyl-2,3-dihydro-1H-inden-2- yl)amino)-1-hydroxyethyl)-8-((4- methylbenzyl)oxy)quinolin-2(1H)-one (I₅) | 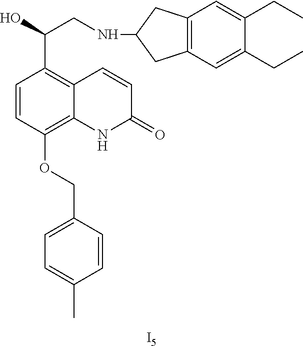 |
| (S)-5-(2-((5,6-diethyl-2,3-dihydro-1H-inden-2-yl)amino)- 1-hydroxyethyl)-8-((3-fluorobenzyl)oxy)quinolin-2(1H)- one (I₆) | 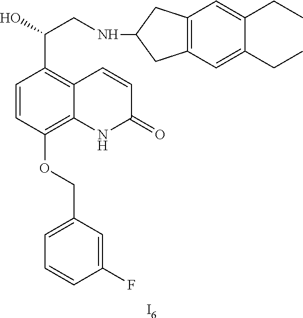 |
| (R)-5-(2-((5,6-diethyl-2,3-dihydro-1H-inden-2- yl)amino)-1-hydroxyethyl)-8-((3- fluorobenzyl)oxy)quinolin-2(1H)-one (I₇) | 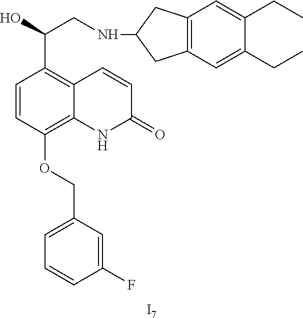 |
| (R)-5-(2-((5,6-diethyl-2,3-dihydro-1H-inden-2- yl)amino)-1-hydroxyethyl)-8-((2- fluorobenzyl)oxy)quinolin-2(1H)-one (I₈) | 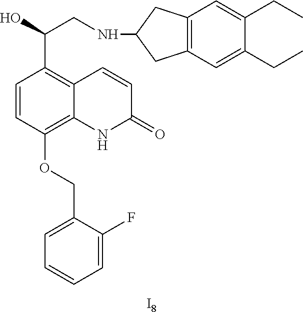 |
| (S)-5-(2-((5,6-diethyl-2,3-dihydro-1H-inden-2-yl)amino)- 1-hydroxyethyl)-8-((3,5-difluorobenzyl)oxy)quinolin- 2(1H)-one (I₉) | 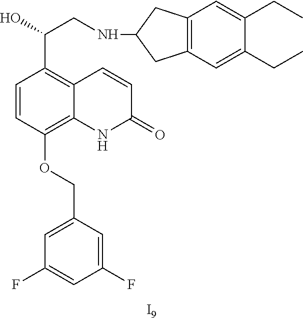 |
| (S)-5-(2-((5,6-diethyl-2,3-dihydro-1H-inden-2-yl)amino)- 1-hydroxyethyl)-8-((3,4-difluorobenzyl)oxy)quinolin- 2(1H)-one (I₁₀) | 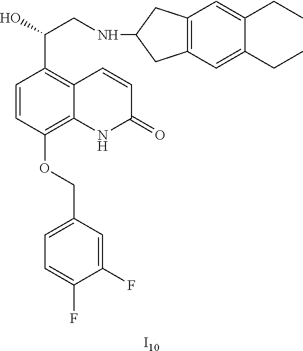 |
| (S)-5-(2-((5,6-diethyl-2,3-dihydro-1H-inden-2-yl)amino)- 1-hydroxyethyl)-8-((perfluorophenyl)methoxy)quinolin- 2(1H)-one (I₁₁) | 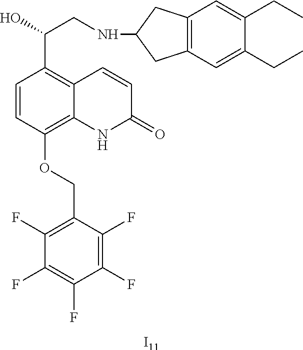 |

The second aspect of the invention provides a method for preparing the compound, including the following steps:

Compound II was obtained by bromination of 5-acetyl-2,8-dihydroxyquinoline after reaction with RIX. The brominating agent selected for bromination could be bromide water, N-bromosuccinimide or tetrabutyltriammonium bromide. Compound II was stereoselectively reduced to compound III by (R)/(S)-2-methyl-CBS-oxazoloborane in combination with borane. Compound III was intramolecular cyclized under alkaline conditions and then reacted with nucleophile R²H to obtain the target compound I. The synthesis route was as follows:

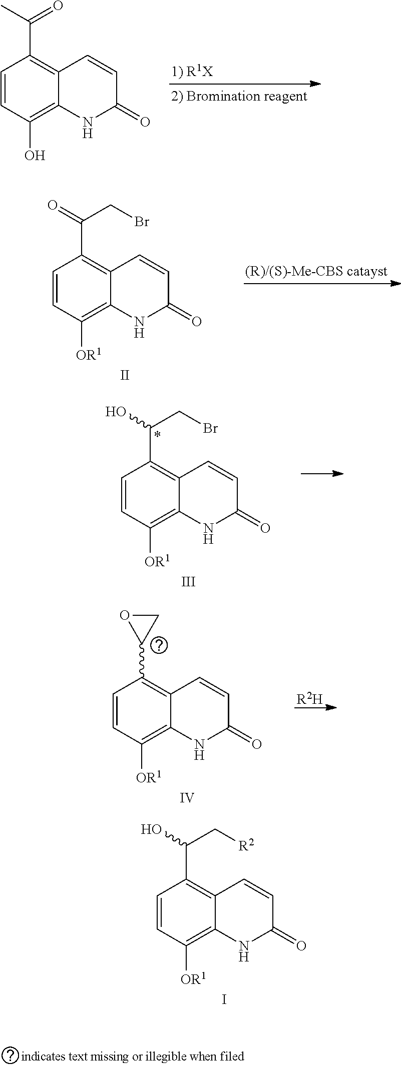

In the reaction for the preparation of compound III from compound II, the solvent is chosen from one or more of anhydrous acetonitrile, anhydrous dichloromethane, chloroform, anhydrous tetrahydrofuran, anhydrous N,N-dimethylformamide, dimethyl sulfoxide. According to the chiral requirements, (R) or (S)-CBS chiral catalysts were selected for asymmetric reduction with borane. The reaction temperature was −20° C. to 25° C.

In the reaction for the preparation of compound IV from compound III: the solvent was chosen from one or more of acetonitrile, dichloromethane, chloroform, acetone, tetrahydrofuran, methanol, N,N-dimethylformamide, dimethyl sulfoxide or dioxane. The base is selected from potassium carbonate, sodium carbonate, sodium bicarbonate, sodium hydroxide, potassium hydroxide or triethylamine; The reaction temperature was 25° C. to reflux;

In the reaction to prepare compound I from compound IV, the solvent was selected from acetonitrile, N,N-dimethylformamide, dimethyl sulfoxide, n-butanol or diethylene glycol dimethyl ether. The reaction temperature was from 60° C. to 150° C.

As a more preferred method for the preparation of the compound, in the reaction of preparation of compound III from compound II, anhydrous tetrahydrofuran is selected as solvent, and (R)-2-methyl-CBS-oxazoloborane or (S)-2-methyl-CBS-oxazoloborane is selected as needed as catalyst, and the reaction temperature is −20° C. to 0° C.

In the reaction for the preparation of compound IV from compound III: acetone was chosen as the solvent, potassium carbonate or triethylamine as the base, and the reaction temperature was 55° C. to 65° C.

In the reaction for the preparation of compound I from compound IV: n-butanol was chosen as the solvent and the reaction temperature was 110° C. to 120° C.

These compounds can be purified according to conventional isolation techniques.

The third aspect of the invention provides the application of the compound in the preparation of drugs for treating diseases related to malignant tumors.

Preferably, the disease associated with malignancy is a variety of tumors mediated by SRSF6 protein overexpression, including colorectal tumors (colorectal tumors mediated by SRSF6 protein overexpression).

In some embodiments of the present invention, using indacaterol and its intermediate as a positive control, the compounds of the present invention have been demonstrated to have a significant inhibitory effect on colorectal cancer cell lines such as RKO, HT-29, and SW620 by in vitro cell experiments.

Beneficial effects: The compounds of the present invention are used for targeted therapy of colorectal tumors and other tumors with high expression of SRSF6, providing potential drug molecules for the chemotherapy of colorectal cancer.

### DESCRIPTION OF THE EMBODIMENTS

In order to further illustrate the present invention, a series of examples are given below, which are entirely illustrative and are intended only for the specific description of the present invention and should not be construed as limiting the present invention.

### PART I: Synthesis of the Compounds

### Example 1: Preparation of (S)-5-(2-((5,6-diethyl-2,3-dihydro-1H-inden-2-yl)amino)-1-hydroxyethyl)-8-((4-fluorobenzyl)oxy)quinolin-2(1H)-one (I₁/LYW2)

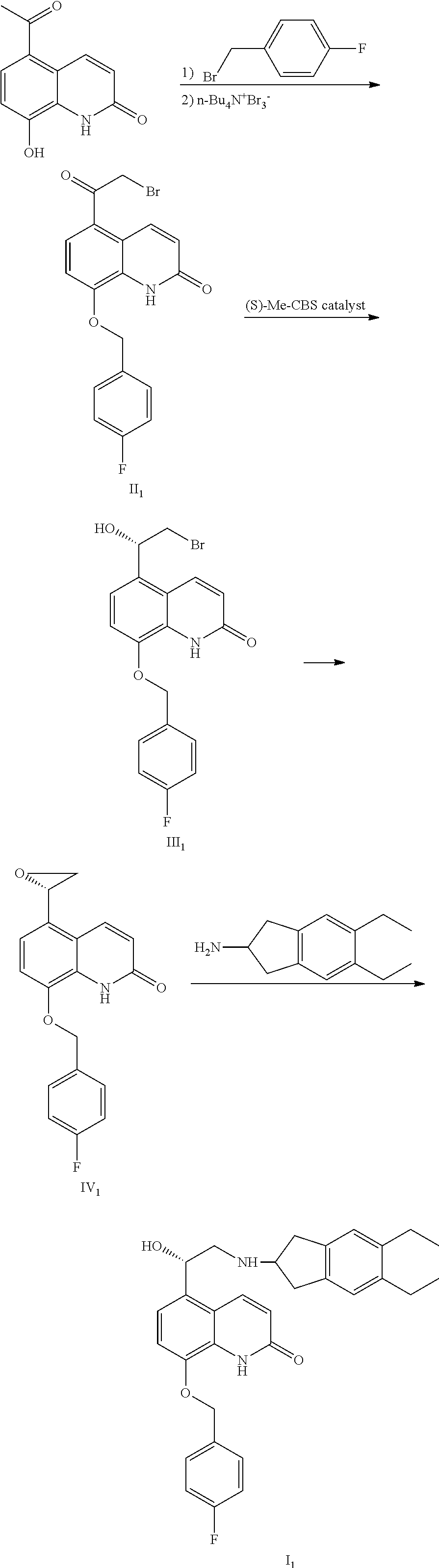

### 1.1 Synthesis of Intermediate II₁

After 5-acetyl-2,8-dihydroxyquinoline (1 g, 4.92 mmol) and anhydrous potassium carbonate (816 mg, 5.91 mmol) were added to the reaction flask, acetone (20 mL) was added and heated up to reflux. Under reflux conditions, 4-fluorobenzyl bromide (727 μL, 5.91 mmol) was added dropwise, and 1.3 g filter cake was collected after stirring.

After the filter cake (1.3 g, 4.18 mmol) was added to the reaction flask, anhydrous THF (26 mL) and anhydrous methanol (10 mL) were added, and the temperature was heated to 40° C. Then tetrabutyltriammonium bromide (2.82 g, 5.85 mmol) was added slowly to brominate the terminal primary carbon. The representative intermediate 5-(2-bromoacetyl)-8-4-(fluorobenzyl) oxyquinoline-2 (1H)-one (compound II₁) was obtained.

¹H NMR (300 MHz, DMSO-d₆) δ 11.14 (s, 1H), 8.50 (d, J=10.1 Hz, 1H), 7.88 (d, J=8.6 Hz, 1H), 7.71-7.65 (m, 2H), 7.32 (d, J=8.6 Hz, 1H), 7.25 (d, J=8.9 Hz, 2H), 6.68 (d, J=10.0 Hz, 1H), 5.41 (s, 2H), 4.94 (s, 2H). MS (ESI) m/z=390.0 [M+H]⁺(C₁₈H₁₃BrFNO₃).

### 1.2 Synthesis of Intermediate III₁

Compound II₁ (500 mg, 1.27 mmol) was added to a 100 mL double-neck reaction flask under nitrogen followed by addition of anhydrous THF (15 mL) and a catalytic amount of (S)-2-methyl-CBS-oxazoloborane (1 mol/L, 128 μL, 0.13 mmol) at −10° C. After 10 min, BH₃-THF (1.54 mL, 1.54 mmol) was slowly added into the reaction system within 0.5 h, and stirred for 15 min. The reaction was monitored by TLC and quenched with methanol (5 mL). After the solvent was removed by reducing pressure concentration, 1 mol/L hydrochloric acid solution (50 mL) was added to the flask, stirred at room temperature overnight and filtered. The white solid III₁ was obtained as residue and dried under vacuum drying, with a yield of 90.53%.

¹H NMR (400 MHz, DMSO-d₆) δ 10.79 (s, 1H), 8.19 (d, J=10.0 Hz, 1H), 7.69-7.62 (m, 2H), 7.27-7.17 (m, 4H), 6.57 (d, J=9.9 Hz, 1H), 5.97 (s, 1H), 5.28 (s, 2H), 5.26-5.18 (m, 1H), 3.69 (dd, J=10.4, 4.8 Hz, 1H), 3.65-3.60 (m, 1H). MS (ESI) m/z=392.0 [M+H]⁺(C₁₈H₁₅BrFNO₃).

### 1.3 Synthesis of Intermediate IV₁

Compound III₁ (400 mg, 1.02 mmol) was added to a 100 mL single-mouthed reaction flask with acetone (40 mL) and anhydrous potassium carbonate (211 mg, 1.53 mmol), and heated to reflux. After 4 h, the reaction was monitored by TLC for completion, then was filtered. The residue washed with acetones and collected. Ethyl acetate was added to recrystallize the crude product to obtain pale yellow solid IV₁ with a yield of 85.67%.

¹H NMR (400 MHz, Chloroform-d) δ 9.23 (s, 1H), 8.11 (d, J=9.8 Hz, 1H), 7.47-7.39 (m, 2H), 7.16-7.08 (m, 3H), 7.02 (d, J=8.3 Hz, 1H), 6.75 (d, J=9.8 Hz, 1H), 5.16 (s, 2H), 4.26-4.21 (m, 1H), 3.23 (dd, J=5.6, 4.0 Hz, 1H), 2.80 (dd, J=5.6, 2.6 Hz, 1H). MS (ESI) m/z=312.1 [M+H]⁺(C₁₈H₁₄FNO₃).

### 1.4 Synthesis of I₁/LYW2

Compound IV₁ (250 mg, 0.80 mmol) and 5,6-diethyl-2,3-dihydro-1H-indenin-2-amine (167 mg, 0.88 mmol) were added to a single-mouth reaction flask, dissolved with 15 mL n-butanol, and then heated to 110° C. for 5 h. The reaction was then concentrated and purified by silica gel column chromatography to obtain I₁ with a yield of 20.15%.

¹H NMR (400 MHz, DMSO-d₆) δ 10.81 (s, 1H), 8.28 (d, J=10.0 Hz, 1H), 7.69-7.63 (m, 2H), 7.27-7.18 (m, 4H), 6.98 (s, 2H), 6.58 (d, J=9.9 Hz, 1H), 5.34 (d, J=8.9 Hz, 1H), 3.82 (p, J=7.4 Hz, 1H), 3.12 (ddd, J=15.6, 11.7, 7.6 Hz, 2H), 3.02-2.82 (m, 4H), 2.57 (d, J=7.5 Hz, 2H), 2.53 (d, J=7.4 Hz, 2H), 1.12 (t, J=7.5 Hz, 6H). ¹³C NMR (101 MHz, DMSO-d₆) δ 161.37, 143.93, 140.07, 138.41, 138.36, 136.91, 132.02, 130.67, 130.59, 129.91, 124.64, 122.81, 119.88, 115.69, 115.48, 112.63, 69.48, 67.19, 58.75, 54.18, 37.28, 25.33, 16.10. MS (ESI) m/z=501.3 [M+H]⁺(C₃₁H₃₃FN₂O₃).

### Example 2: Preparation of (R)-5-(2-((5,6-diethyl-2,3-dihydro-1H-inden-2-yl)amino)-1-hydroxyethyl)-8-((4-fluorobenzyl)oxy)quinolin-2(1H)-one (I₂)

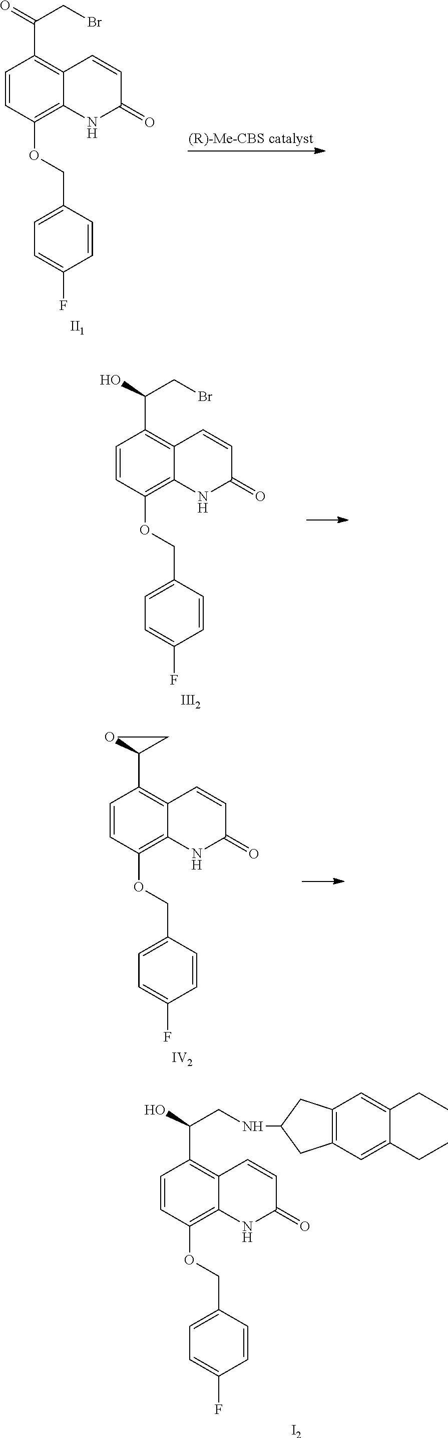

### 2.1 Synthesis of Intermediate III₂

In the preparation of compound III from compound II, the catalyst in the reaction is changed to (R)-2-methyl-CBS-oxazolaborane, that is, the enantiomer III₂ of intermediate III₁ in Example 1, as follows:

Compound II₁ (500 mg, 1.27 mmol) was added to a 100 mL double-neck reaction flask under nitrogen with anhydrous THF (15 mL) and a catalytic amount of (R)-2-methyl-CBS-oxazoloborane (1 mol/L, 128 μL, 0.13 mmol) at −10° C. After 10 min, BH₃-THF (1.54 mL, 1.54 mmol) was slowly added into the reaction system within 0.5 hour, and stirred for 15 min. The reaction was monitored by TLC and quenched with methanol (5 mL). After the solvent was removed under reduced pressure, 1 mol/L hydrochloric acid solution (50 mL) was added to the flask and stirred at room temperature overnight and filtered. The residue was collected and dried under vacuum to obtain III₂ with a yield of 91.73%.

¹H NMR (400 MHz, DMSO-d₆) δ 10.78 (s, 1H), 8.19 (d, J=10.0 Hz, 1H), 7.67-7.63 (m, 2H), 7.24-7.19 (m, 4H), 6.57 (d, J=9.9 Hz, 1H), 5.97 (d, J=4.7 Hz, 1H), 5.29 (s, 2H), 5.23 (dt, J=7.3, 4.8 Hz, 1H), 3.69 (dd, J=10.4, 4.8 Hz, 1H), 3.62 (dd, J=10.4, 7.2 Hz, 1H). MS (ESI) m/z=392.0 [M+H]⁺(C₁₈H₁₅BrFNO₃).

### 2.2 Synthesis of I₂

Intermediate III₂ was synthesized according to the method described in step 1.3 of Example 1, and intermediate IV₂ was synthesized according to the method described in step 1.4 of Example 1. The intermediate IV₂ (300 mg, 0.96 mmol) reacted with 5,6-diethyl-2,3-dihydro-1H-indan-2-amine (200 mg, 1.06 mmol) and the reaction was purified by silica gel column chromatography to obtain the I₂ with a yield of 21.35%.

¹H NMR (400 MHz, DMSO-d₆) δ 10.85 (s, 1H), 8.32 (t, J=11.1 Hz, 1H), 7.65 (t, J=6.9 Hz, 2H), 7.33-7.12 (m, 4H), 6.96 (d, J=14.3 Hz, 2H), 6.59 (t, J=7.8 Hz, 1H), 5.48 (s, 1H), 5.29 (s, 2H), 4.04-3.82 (m, 2H), 3.06 (ddd, J=35.7, 20.1, 9.8 Hz, 6H), 2.54 (d, J=7.4 Hz, 3H), 1.10 (t, J=6.7 Hz, 6H). ¹³C NMR (101 MHz, DMSO-d₆) δ 161.41, 144.09, 140.30, 137.78, 133.26, 131.28, 130.66, 130.58, 129.91, 124.56, 122.96, 119.93, 117.08, 115.69, 115.48, 112.60, 69.46, 58.42, 36.11, 25.31, 16.04. MS (ESI) m/z=501.3 [M+H]⁺(C₃₁H₃₃FN₂O₃).

### Example 3: Preparation of (S)-8-((4-fluorobenzyl)oxy)-5-(1-hydroxy-2-methoxyethyl)quinolin-2(1H)-one (I₃)

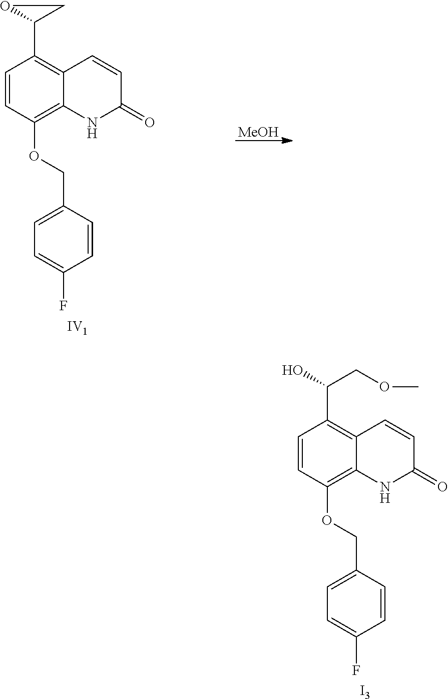

Accodring to the method of step 1.4 in Example 1, the intermediate IV₁ (200 mg) reacted with absolute methanol (2 mL), and the reaction was purification by silica gel column chromatography. I₃ was obtained with a yield of 58.03%.”

¹H NMR (400 MHz, DMSO-d₆) δ 10.70 (s, 1H), 8.21 (d, J=9.9 Hz, 1H), 7.66 (dd, J=8.5, 5.7 Hz, 2H), 7.26-7.19 (m, 3H), 7.06 (d, J=8.3 Hz, 1H), 6.56 (d, J=9.9 Hz, 1H), 5.27 (s, 2H), 4.89 (t, J=5.9 Hz, 1H), 4.65 (dd, J=7.0, 4.6 Hz, 1H), 3.65-3.57 (m, 1H), 3.50-3.42 (m, 1H), 3.19 (s, 3H). ¹³C NMR (101 MHz, DMSO-d₆) δ 161.35, 144.05, 137.22, 133.34, 133.31, 130.69, 130.60, 129.95, 128.99, 122.55, 121.02, 118.22, 115.70, 115.49, 112.46, 82.59, 69.56, 66.08, 56.88. MS (ESI) m/z=344.1 [M+H]⁺(C₁₉H₁₈FNO₄).

### Example 4: Preparation of (S)-5-(2-((5,6-diethyl-2,3-dihydro-1H-inden-2-yl)amino)-1-hydroxyethyl)-8-((4-methylbenzyl)oxy)quinolin-2(1H)-one (I₄)

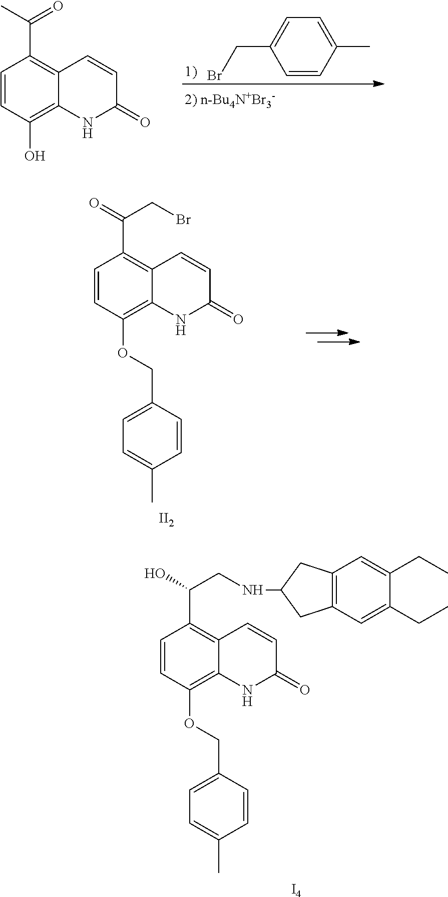

By replacing 4-fluorobromobenzyl in Example 1 with 4-methylbromobenzyl, 14 can be obtained according to the method described in Example 1.

¹H NMR (300 MHz, Chloroform-d) δ 9.38 (s, 1H), 8.48 (s, 1H), 8.08 (d, J=10.0 Hz, 1H), 7.34-7.28 (m, 4H), 7.21 (d, J=7.7 Hz, 2H), 6.96 (d, J=5.1 Hz, 2H), 6.57 (d, J=9.6 Hz, 1H), 5.62 (d, J=9.5 Hz, 1H), 5.12 (d, J=5.1 Hz, 2H), 3.97-3.83 (m, 1H), 3.29-3.05 (m, 6H), 2.59 (q, J=7.5 Hz, 4H), 2.39 (s, 3H), 1.18 (t, J=7.5 Hz, 6H). ¹³C NMR (75 MHz, Chloroform-d) δ 169.64, 161.33, 141.06, 138.51, 136.59, 136.17, 132.46, 129.50, 128.90, 128.01, 124.41, 122.64, 119.96, 111.04, 77.26, 70.89, 66.23, 58.97, 53.74, 36.54, 25.45, 21.28, 15.48. MS (ESI) m/z=497.3 [M+H]⁺(C₃₂H₃₆N₂O₃).

### Example 5: Preparation of (R)-5-(2-((5,6-diethyl-2,3-dihydro-1H-inden-2-yl)amino)-1-hydroxyethyl)-8-((4-methylbenzyl)oxy)quinolin-2(1H)-one (I₅)

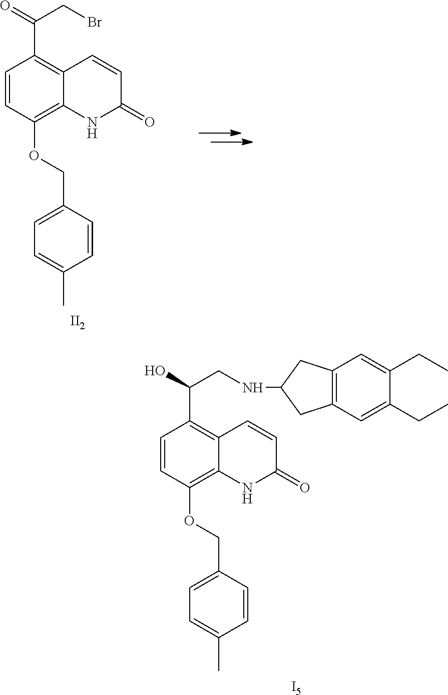

By replacing 4-fluorobromobenzyl in Example 1 with 4-methylbromobenzyl, I₅ can be obtained according to the methods described in Example 2.

¹H NMR (300 MHz, DMSO-d6) δ 8.20 (d, J=10.0 Hz, 1H), 7.45 (d, J=8.0 Hz, 2H), 7.18 (d, J=7.9 Hz, 4H), 6.93 (d, J=2.1 Hz, 2H), 6.54 (d, J=9.9 Hz, 1H), 5.24 (s, 2H), 5.04 (t, J=6.1 Hz, 1H), 3.54-3.49 (m, 1H), 2.98 (m, 2H), 2.73 (d, J=6.4 Hz, 2H), 2.64-2.57 (m, 2H), 2.53 (m, 4H), 2.28 (s, 3H), 1.12 (t, J=7.5 Hz, 6H). ¹³C NMR (75 MHz, DMSO) δ 161.29, 143.59, 139.73, 139.67, 139.44, 137.57, 134.12, 133.47, 129.78, 129.36, 128.37, 124.63, 122.31, 119.84, 117.26, 112.59, 70.12, 69.41, 59.67, 56.03, 40.26, 25.34, 21.24, 16.15. MS (ESI) m/z=497.3 [M+H]⁺(C₃₂H₃₆N₂O₃).

### Example 6: Preparation of (S)-5-(2-((5,6-diethyl-2,3-dihydro-1H-inden-2-yl)amino)-1-hydroxyethyl)-8-((3-fluorobenzyl)oxy)quinolin-2(1H)-one (I₆)

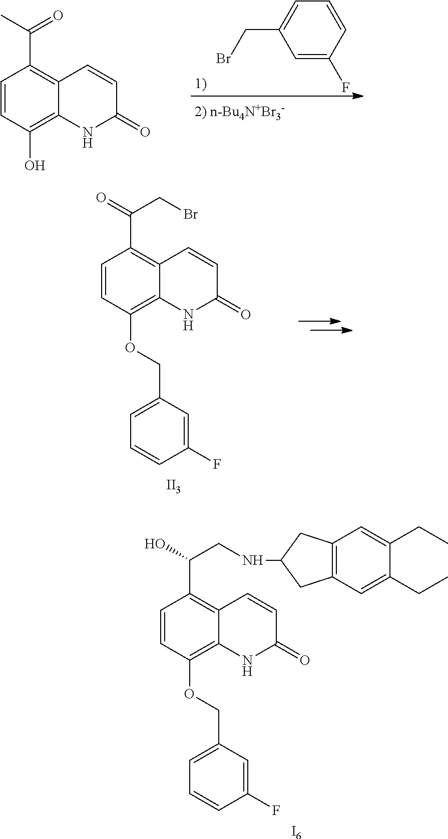

By replacing 4-fluorobromobenzyl in Example 1 with 3-fluorobromobenzyl, 16 can be obtained according to the method described in Example 1.

¹H NMR (400 MHz, DMSO-d₆) δ 10.82 (s, 1H), 8.32 (s, 1H), 8.24 (d, J=9.9 Hz, 1H), 7.54 (d, J=8.7 Hz, 1H), 7.42 (t, J=5.6 Hz, 2H), 7.23 (s, 2H), 7.14 (ddt, J=9.0, 6.2, 2.7 Hz, 1H), 6.97 (s, 2H), 6.59 (d, J=9.9 Hz, 1H), 5.32 (s, 2H), 5.25 (dd, J=9.2, 3.6 Hz, 1H), 3.77 (p, J=7.3 Hz, 1H), 3.09 (ddd, J=15.7, 10.8, 7.5 Hz, 2H), 2.95 (td, J=13.7, 12.9, 6.2 Hz, 2H), 2.83 (dt, J=16.2, 8.0 Hz, 2H), 2.59-2.52 (m, 4H), 1.12 (t, J=7.5 Hz, 6H). ¹³C NMR (101 MHz, DMSO-d₆) δ 165.46, 161.43, 143.83, 139.95, 138.66, 138.62, 136.96, 132.48, 130.76, 130.68, 129.87, 124.64, 124.62, 124.14, 124.11, 122.75, 119.91, 117.22, 115.17, 114.98, 114.95, 112.54, 69.43, 67.61, 58.88, 54.45, 37.75, 37.68, 25.33, 16.09. MS (ESI) m/z=501.3 [M+H]⁺(C₃₁H₃₃FN₂O₃).

### Example 7: Preparation of (R)-5-(2-((5,6-diethyl-2,3-dihydro-1H-inden-2-yl)amino)-1-hydroxyethyl)-8-((3-fluorobenzyl)oxy)quinolin-2(1H)-one (7)

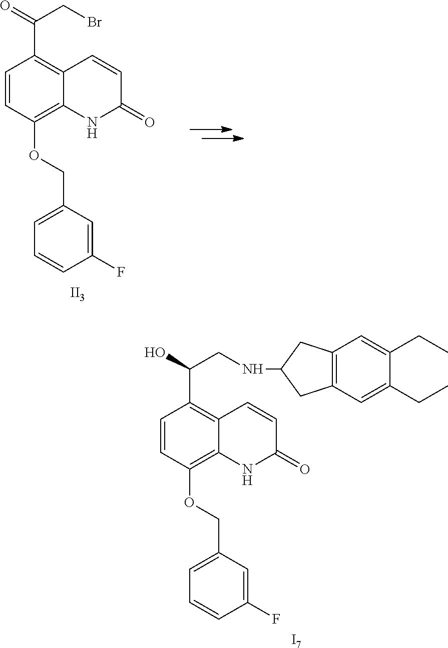

Replacing 4-fluorobromobenzyl in Example 1 with 3-fluorobromobenzyl, I₇ was obtained according to the methods in Example 2.

¹H NMR (400 MHz, DMSO-d₆) δ 10.77 (s, 1H), 8.21 (d, J=10.0 Hz, 1H), 7.57-7.52 (m, 1H), 7.45-7.39 (m, 2H), 7.19 (d, J=2.0 Hz, 2H), 7.14 (ddd, J=9.0, 6.6, 2.6 Hz, 1H), 6.93 (d, J=3.5 Hz, 2H), 6.56 (d, J=9.9 Hz, 1H), 5.42 (s, 1H), 5.31 (s, 2H), 5.05 (t, J=6.1 Hz, 1H), 3.52 (q, J=6.6 Hz, 1H), 2.98 (ddd, J=15.5, 11.6, 6.9 Hz, 2H), 2.77-2.72 (m, 2H), 2.60 (t, J=5.6 Hz, 1H), 2.54 (t, J=7.5 Hz, 5H), 1.12 (t, J=7.5 Hz, 6H). ¹³C NMR (101 MHz, DMSO-d₆) δ 161.46, 143.52, 139.80, 139.75, 139.42, 137.29, 133.84, 130.75, 130.66, 129.77, 124.68, 124.64, 124.12, 124.09, 122.40, 119.86, 117.37, 115.17, 114.95, 112.48, 69.52, 69.43, 59.72, 56.13, 25.34, 16.14. MS (ESI) m/z=501.3 [M+H]⁺(C₃₁H₃₃FN₂O₃).

### Example 8: Preparation of (R)-5-(2-((5,6-diethyl-2,3-dihydro-1H-inden-2-yl)amino)-1-hydroxyethyl)-8-((2-fluorobenzyl)oxy)quinolin-2(1H)-one (I₈)

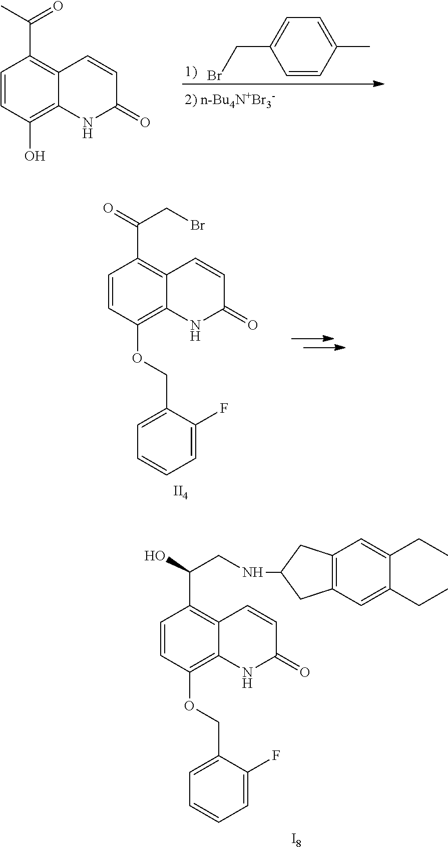

By replacing 4-fluorobromobenzyl in Example 1 with 2-fluorobromobenzyl, the target product I₈ can be obtained according to the methods and reaction conditions in Example 2.

¹H NMR (300 MHz, DMSO-d₆) δ 10.62 (s, 1H), 8.42 (d, J=10.0 Hz, 1H), 7.72 (t, J=7.6 Hz, 1H), 7.46-7.34 (m, 2H), 7.26 (dd, J=14.3, 7.7 Hz, 3H), 6.91 (d, J=5.8 Hz, 2H), 6.59 (d, J=9.9 Hz, 1H), 5.31 (s, 2H), 5.06 (s, 1H), 4.48 (s, 1H), 3.68-3.49 (m, 4H), 2.86 (dp, J=20.5, 6.9 Hz, 3H), 2.54 (d, J=7.9 Hz, 4H), 1.09 (t, J=7.5 Hz, 6H). ¹³C NMR (75 MHz, DMSO-d₆) δ 161.33, 143.86, 139.67, 136.95, 131.34, 131.29, 130.88, 130.77, 129.80, 124.97, 124.92, 124.71, 124.48, 123.98, 123.79, 122.57, 121.50, 118.68, 115.88, 115.60, 112.59, 64.80, 57.65, 25.30, 16.08. MS (ESI) m/z=501.3 [M+H]⁺(C₃₁H₃₃FN₂O₃).

### Example 9: Preparation of (S)-5-(2-((5,6-diethyl-2,3-dihydro-1H-inden-2-yl)amino)-1-hydroxyethyl)-8-((3,5-difluorobenzyl)oxy)quinolin-2(1H)-one (I₉)

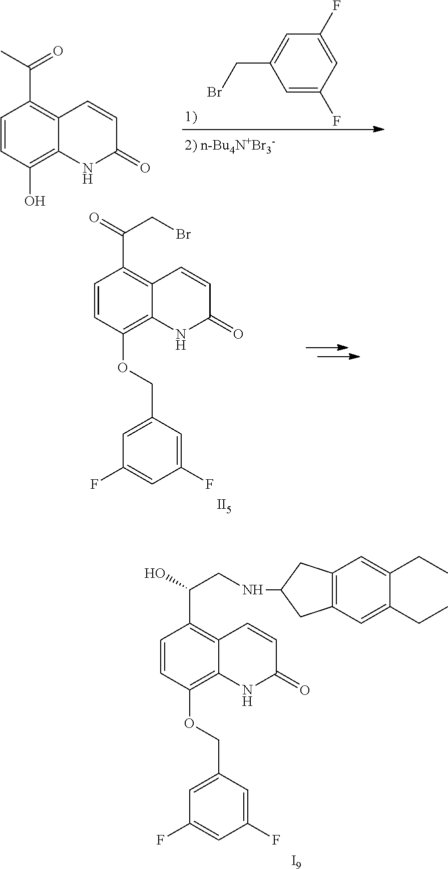

By replacing 4-fluorobrobenzyl in Example 1 with 3, 5-difluorobrobenzyl, the target product I₉ can be obtained according to the method and reaction conditions in Example 1.

¹H NMR (400 MHz, DMSO-d₆) δ 11.01 (s, 1H), 8.34-8.30 (m, 1H), 8.24 (d, J=9.7 Hz, 1H), 7.44 (d, J=6.8 Hz, 2H), 7.23 (s, 2H), 7.17 (dt, J=9.1, 2.4 Hz, 1H), 6.97 (s, 2H), 6.59 (d, J=9.9 Hz, 1H), 5.31 (s, 2H), 5.22 (s, 1H), 3.77-3.67 (m, 1H), 3.07 (ddd, J=15.3, 11.2, 7.3 Hz, 2H), 2.89 (p, J=11.5 Hz, 2H), 2.78 (dt, J=15.2, 7.0 Hz, 2H), 2.55 (t, J=7.5 Hz, 4H), 1.12 (t, J=7.5 Hz, 6H). ¹³C NMR (101 MHz, DMSO-d₆) δ 161.52, 143.64, 139.84, 138.85, 136.98, 129.80, 124.64, 122.76, 119.89, 117.28, 112.45, 111.39, 111.14, 103.63, 68.92, 67.92, 59.04, 54.74, 25.33, 16.12. MS (ESI) m/z=519.3 [M+H]⁺(C₃₁H₃₂F₂N₂O₃).

### Example 10: Preparation of (S)-5-(2-((5,6-diethyl-2,3-dihydro-1H-inden-2-yl)amino)-1-hydroxyethyl)-8-((3,4-difluorobenzyl)oxy)quinolin-2(1H)-one (I₁₀)

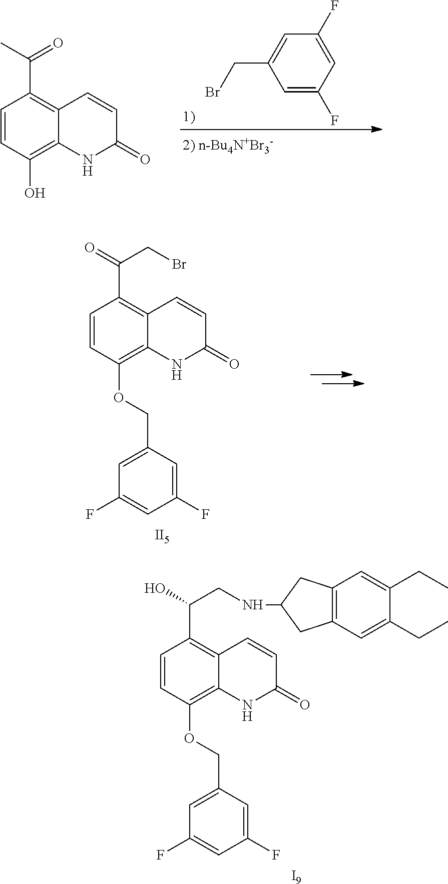

By replacing 4-fluorobrobenzyl in Example 1 with 3, 4-difluorobrobenzyl, the target product I₁₀ can be obtained according to the method and reaction conditions in Example 1.

¹H NMR (400 MHz, DMSO-d₆) δ 10.87 (s, 1H), 8.23 (d, J=10.0 Hz, 1H), 7.85-7.79 (m, 1H), 7.44 (q, J=4.5 Hz, 2H), 7.27-7.19 (m, 2H), 6.95 (d, J=2.0 Hz, 2H), 6.57 (d, J=9.9 Hz, 1H), 5.27 (s, 2H), 5.17 (dd, J=8.1, 4.4 Hz, 1H), 3.64 (q, J=7.0 Hz, 1H), 3.04 (ddd, J=15.3, 11.3, 7.2 Hz, 2H), 2.85 (q, J=7.2, 6.2 Hz, 2H), 2.72 (ddd, J=15.7, 6.9, 3.8 Hz, 2H), 2.55 (t, J=7.5 Hz, 4H), 1.12 (t, J=7.5 Hz, 6H). ¹³C NMR (101 MHz, DMSO-d₆) δ 161.49, 143.62, 139.71, 139.16, 139.12, 137.09, 134.91, 133.15, 125.20, 124.67, 124.63, 122.62, 119.86, 117.79, 117.62, 112.50, 68.94, 68.40, 59.23, 55.21, 38.64, 38.52, 25.33, 16.13. MS (ESI) m/z=519.3 [M+H]⁺(C₃₁H₃₂F₂N₂O₃).

### Example 11: Preparation of (S)-5-(2-((5,6-diethyl-2,3-dihydro-1H-inden-2-yl)amino)-1-hydroxyethyl)-8-((perfluorophenyl)methoxy)quinolin-2(1H)-one (I₁₁)

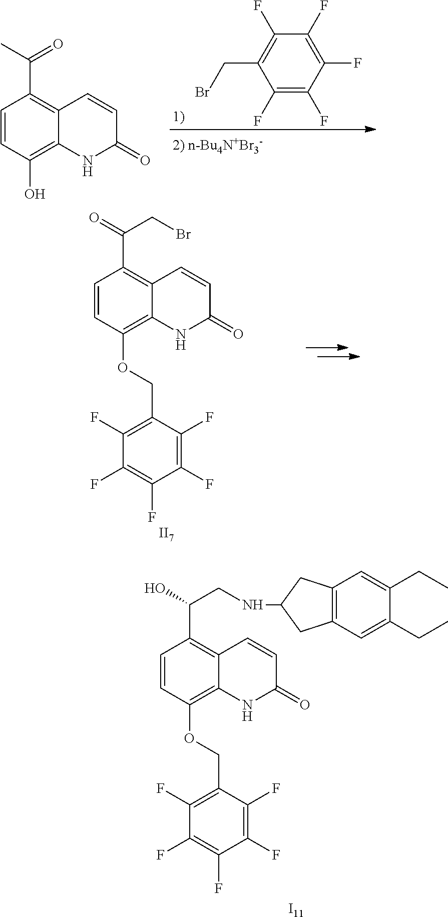

By replacing 4-fluorobromobenzyl in Example 1 with perfluorobromobenzyl, the target product I₁₁ can be obtained according to the method and reaction conditions in Example 1.

¹H NMR (400 MHz, DMSO-d₆) δ 10.84 (s, 1H), 8.21 (d, J=10.0 Hz, 1H), 7.32 (d, J=8.4 Hz, 1H), 7.25 (d, J=8.4 Hz, 1H), 6.95 (d, J=3.8 Hz, 2H), 6.54 (d, J=9.9 Hz, 1H), 5.28 (s, 2H), 5.08 (t, J=6.1 Hz, 1H), 3.54 (q, J=6.7 Hz, 1H), 2.99 (ddd, J=15.4, 11.7, 7.0 Hz, 2H), 2.76 (d, J=6.2 Hz, 2H), 2.65-2.57 (m, 2H), 2.54 (d, J=7.5 Hz, 4H), 1.12 (t, J=7.5 Hz, 6H). ¹³C NMR (101 MHz, DMSO-d₆) δ 161.54, 143.71, 139.77, 139.45, 137.19, 134.41, 129.52, 124.71, 124.66, 122.57, 119.87, 117.31, 112.21, 69.44, 59.71, 58.48, 56.13, 25.34, 16.16. (ESI) m/z=573.3 [M+H]⁺(C₃₁H₂₉F₅N₂O₃).

### PART II: Inhibitory Effect of Compound I on Colorectal Cancer Cell Lines

Based on the therapeutic effect of indacaterol, we evaluated LYW1 as the lead compound and investigated the in vitro inhibitory effect of series I compound on colorectal cancer cell lines. It was found that they had a strong inhibitory effect on RKO, HT-29, and SW620. The specific experimental process and results are as follows:

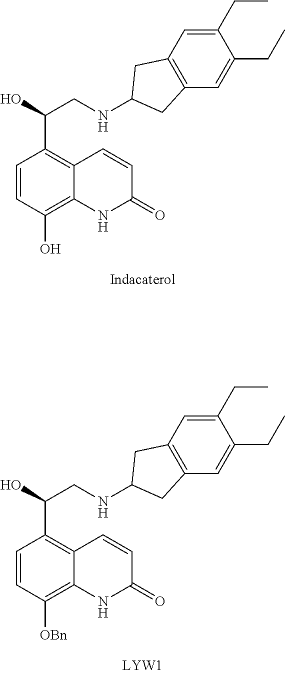 [SMILES: CCc1cc2c(cc1CC)CC(NC[C@H](O)c1ccc(O)c3[nH]c(=O)ccc13)C2 ; CCc1cc2c(cc1CC)CC(NC[C@H](O)c1ccc(OCc3ccccc3)c3[nH]c(=O)ccc13)C2]

### 1. Inhibitory Effect of Compound I on SW620 Cells:

Concentration gradients of 10, 20, 30, and 40 μM were prepared using indacaterol as a positive control, and the IC₅₀ for SW620 was fixed between 20 and 25 μM. Compound I was prepared with the same concentration gradient for primary screening, and compounds with lower IC₅₀ than indacaterol were screened for secondary screening with concentration gradients of 1, 2, 4, and 8 μM.

Both rounds of screening were performed on the colorectal cancer cell line SW620 with an initial cell number of 5000 cells per well, and the cell survival rate at 48 h was determined directly to determine the IC₅₀ of the compound.

Cell viability=[(reading of experimental group−reading of blank group)/(reading of control group−reading of blank group)]×100%.

TABLE 1
The inhibitory effects on SW620 cells

| Compound | IC₅₀ (μM) |
|---|---|
| Indacaterol | 23.05 |
| LYW1 | 3.542 |
| I₁ | 3.121 |
| I₂ | 8.624 |
| I₄ | 2.899 |
| I₅ | 2.975 |
| I₆ | 3.055 |
| I₇ | 2.734 |
| I₉ | 3.154 |
| I₁₀ | 4.184 |
| I₁₁ | 6.093 |

### 2. Inhibitory Effect of Compound I on RKO (Human Colon Adenocarcinoma Cells) and HT29 (Human Colon Cancer Cells):

Indacaterol was used as a positive control, and its IC₅₀ for RKO, HT₂₉ was fixed at 10-15 μM, and the other procedures were the same as for SW620 cell line.

TABLE 2
The inhibitory effects on RKO cells

| Compound | IC₅₀ (μM) |
|---|---|
| Indacaterol | 12.79 |
| LYW1 | 2.033 |
| I₁ | 1.422 |
| I₂ | 5.563 |
| I₇ | 3.107 |

TABLE 3
The inhibitory effects on HT29 cells

| Compound | IC₅₀ (μM) |
|---|---|
| Indacaterol | 12.71 |
| LYW1 | 2.425 |
| I₁ | 1.422 |
| I₂ | 4.687 |
| I₇ | 2.663 |

### PART III. Studies on the Binding of Compound I₁/LYW2 to SRSF6 Protein

SRSF proteins play an important role in the regulation of gene expression, including constitutive splicing, alternative splicing, mRNA nuclear export and translation. SRSF proteins can be divided into two groups according to the number of RRM domains. Among them, SRSF6 has RRM1 and RRM2 domains. The RRM1 domain contains conserved sequences of RNP-1 and RNP-2, which can bind to related mRNAs. However, the RRM2 domain contains the evolutionarily conserved SWQDLKD heptapeptide sequence. RRM1 and RRM2 domains play irreplaceable roles in the specific recognition of alternative splice sites. Overexpression of SRSF6 induced colorectal carcinogenesis by binding to cell adhesion factor ZO-1, while indacaterol was validated to inhibit tumor development. Therefore, RRM1 and RRM2 domains of SRSF6 were extracted and the binding ability of compound I₁ to SRSF6 was investigated by protein thermal shift assay using LYW1 as a reference.

We used DMSO as the blank group, and 56.5 μM SRSF6 protein was added to the PCR tube with LYW1 and I₁ at a concentration of 1:10, respectively. After TS dye was added in the dark, the thermal migration assay was performed. The experimental results are as follows:

TABLE 4
Results of SRSF6 protein thermal shift assay

| # | Well | Protein | Ligand | Task | ΔTₘ B (° C.) | ΔTₘ D (° C.) |
|---|---|---|---|---|---|---|
| 1 | A01 | SRSF6 | DMSO | R | 0.01 | −0.07 |
| 2 | B01 | SRSF6 | DMSO | R | −0.01 | 0.07 |
| 3 | C01 | SRSF6 | LYW1 | S | −14.11 | −13.66 |
| 4 | D01 | SRSF6 | LYW1 | S | −13.12 | −12.82 |
| 5 | E01 | SRSF6 | I₁/LYW2 | S | −10.35 | −10.16 |
| 6 | F01 | SRSF6 | I₁/LYW2 | S | −11.58 | −10.86 |

The experimental results showed that compared with DMSO, LYW1 and I₁ both caused different degrees of negative migration of SRSF6 protein, but LYW1 itself had a strong fluorescence, so I₁ showed a better binding effect with SRSF6 protein.

What is claimed is:

1. In one aspect, compounds or pharmaceutically acceptable salts thereof useful as SRSF6 modulators or inhibitors having the structure of Formula I are described herein, 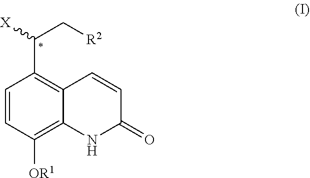
where R¹ is selected from a substituted or unsubstituted C1-C3 linear alkyl group, a substituted or unsubstituted C1-C3 alkenyl group, a substituted or unsubstituted C6-C10 aryl group, a substituted or unsubstituted C6-C10 aralkyl group, a C6-C10 haloalkyl group, or a C6-C10 heteroaryl group, wherein the heteroatoms of the heteroaryl group are selected independently from N, O, or S;
R² is selected from a C1-C3 alkoxy group, substituted amino group, C1-C8 aliphatic amine group, saturated or unsaturated substituted or unsubstituted C6-C10 aryl group, substituted or unsubstituted C6-C10 heteroaryl group, halogenated aromatic carbocyclic amine group or substituted or unsubstituted 5- to 7-membered heterocyclic group; wherein, the heteroatoms in the heteroaryl group or heterocyclic group are independently selected from N, O, or S, and the halogen atoms refer to fluorine, chlorine, bromine, or iodine; the substituted amino group is replaced by one to three of the following substituents: C1-C4 alkyl, 5,6-diethyl indenyl, substituted or unsubstituted phenyl, indenyl, indolyl, methylsulfonyl substituted piperidinyl, benzothiazolyl, benzimidazolyl, respectively;
X is selected from a hydroxyl group or a halogen, and the halogen is selected from fluorine, chlorine, bromine and iodine;
the chiral center * is the S configuration or the R configuration.

2. The compound of claim 1, wherein R¹ is selected from a methyl group, an allyl group, a substituted or unsubstituted benzyl group, a substituted or unsubstituted pyridyl group, or a triazole; The substituted or unsubstituted benzyl group is independently substituted by one to three of the following groups: a halogen or a C1-C3 alkyl group;
R² is selected from methoxy, 5,6-diethyl-2,3-dihydro-1H-indenin-2-amino, 2-aminoindenyl, aniline, bromoaniline, heteroarylamine, C1-C4 aliphatic amine, morpholinyl, piperidinyl, pyrrolidine, substituted or unsubstituted piperazinyl, substituted or unsubstituted hyperpiperazinyl, indolyl, 2,3-dihydroindenyl; the substituted or unsubstituted piperazinyl group or the substituted or unsubstituted hyperpiperazinyl group is replaced by one to three independent substituents: C1-C4 alkyl group, substituted or unsubstituted phenyl group, or substituted or unsubstituted benzyl group, wherein the phenyl or benzyl group is replaced by a halogen or C1-C3 alkyl group.

3. The compound of claim 1, wherein R¹ is selected from benzyl, 3-substituted benzyl, 4-substituted benzyl or multi-substituted benzyl, and the benzyl substituent is a methyl group or a halogen;
R² is selected from 5,6-diethyl-2,3-dihydro-1H-indenin-2-amino, N-phenylpiperazinyl, N-phenylhomopiperazinyl, bromoanilyl, 2,3-dihydroindolyl or 2,3-dihydroindenyl; The N-phenylpiperazinyl is selected from: benzyl substituted piperazinyl, methyl benzyl substituted piperazinyl or benzyl bromide substituted piperazinyl; N-benzyl hyperpiperazinyl is selected from: benzyl substituted for hyperpiperazinyl, methyl benzyl substituted for hyperpiperazinyl, or benzyl bromide substituted for hyperpiperazinyl.

4. The compound of claim 1, wherein the R¹ is selected from benzyl, 4-methylbenzyl, 3-fluorobenzyl, 4-fluorobenzyl, 3,5-difluorobenzyl, 3,4-difluorobenzyl, perfluorobenzyl;
R² was selected from 5,6-diethyl-2,3-dihydro-1H-indenin-2-amine, 2-bromoaniline, 2,3-dihydroindolyl, 2,3-dihydroindenyl, benzyl-substituted piperazinyl, 4-methylbenzyl-substituted piperazinyl, 4-bromobenzyl-substituted piperazinyl, benzyl-substituted high-piperazinyl, 4-methylbenzyl-substituted high-piperazinyl.

5. The compound of claim 1, wherein the compound is:

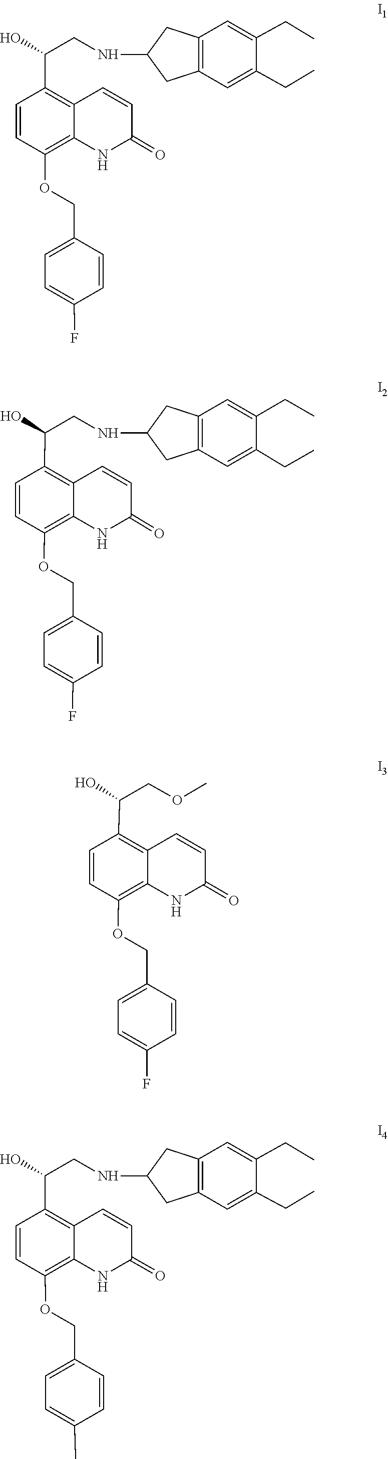 [SMILES: CCc1cc2c(cc1CC)CC(NC[C@@H](O)c1ccc(OCc3ccc(F)cc3)c3[nH]c(=O)ccc13)C2 ; CCc1cc2c(cc1CC)CC(NC[C@H](O)c1ccc(OCc3ccc(F)cc3)c3[nH]c(=O)ccc13)C2 ; COC[C@@H](O)c1ccc(OCc2ccc(F)cc2)c2[nH]c(=O)ccc12 ; CCc1cc2c(cc1CC)CC(NC[C@@H](O)c1ccc(OCc3ccc(C)cc3)c3[nH]c(=O)ccc13)C2]

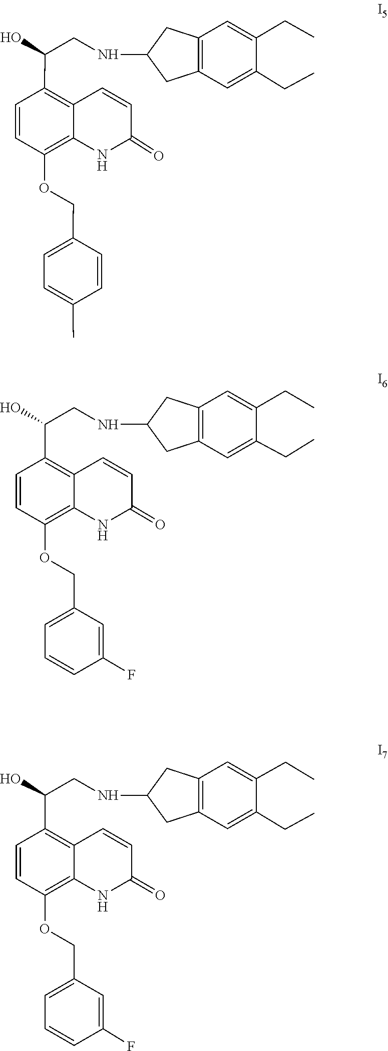 [SMILES: CCc1cc2c(cc1CC)CC(NC[C@H](O)c1ccc(OCc3ccc(C)cc3)c3[nH]c(=O)ccc13)C2 ; CCc1cc2c(cc1CC)CC(NC[C@@H](O)c1ccc(OCc3cccc(F)c3)c3[nH]c(=O)ccc13)C2 ; CCc1cc2c(cc1CC)CC(NC[C@H](O)c1ccc(OCc3cccc(F)c3)c3[nH]c(=O)ccc13)C2]

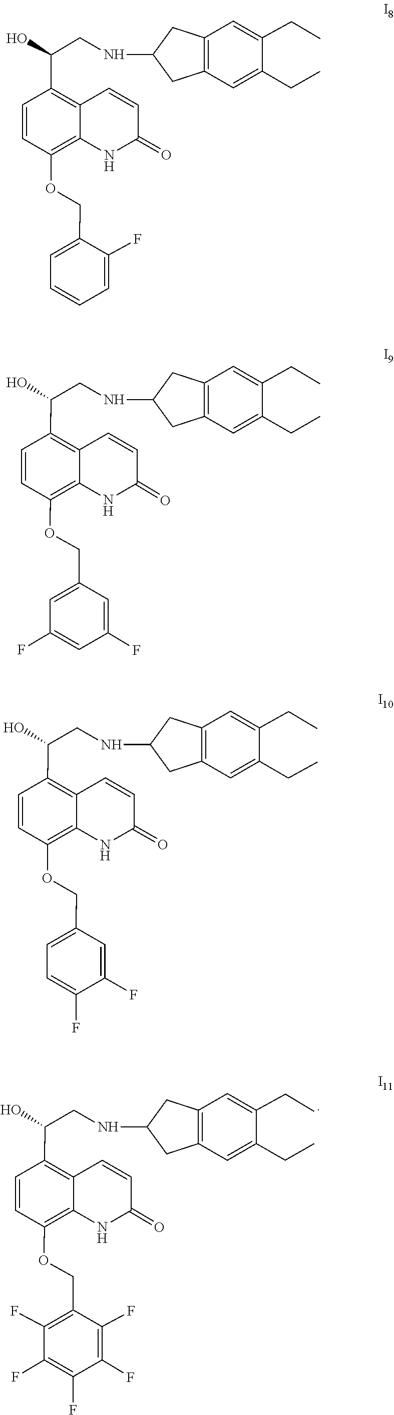 [SMILES: CCc1cc2c(cc1CC)CC(NC[C@H](O)c1ccc(OCc3ccccc3F)c3[nH]c(=O)ccc13)C2 ; CCc1cc2c(cc1CC)CC(NC[C@@H](O)c1ccc(OCc3cc(F)cc(F)c3)c3[nH]c(=O)ccc13)C2 ; CCc1cc2c(cc1CC)CC(NC[C@@H](O)c1ccc(OCc3ccc(F)c(F)c3)c3[nH]c(=O)ccc13)C2 ; CCc1cc2c(cc1CC)CC(NC[C@@H](O)c1ccc(OCc3c(F)c(F)c(F)c(F)c3F)c3[nH]c(=O)ccc13)C2]

6. A method for preparing the compound according to claim 1, comprising the following steps:
compound II was obtained by bromination of 5-acetyl-2,8-dihydroxyquinoline with R¹X; bromination agents were selected from brominated water, N-bromosuccinimide or tetrabutyltriammonium bromide; compound II was stereoselectively reduced to compound III by (R)/(S)-2-methyl-CBS-oxazoloborane in combination with borane, which then was intramolecular cyclized under alkaline conditions and reacted with nucleophile R²H to give the target compound I:

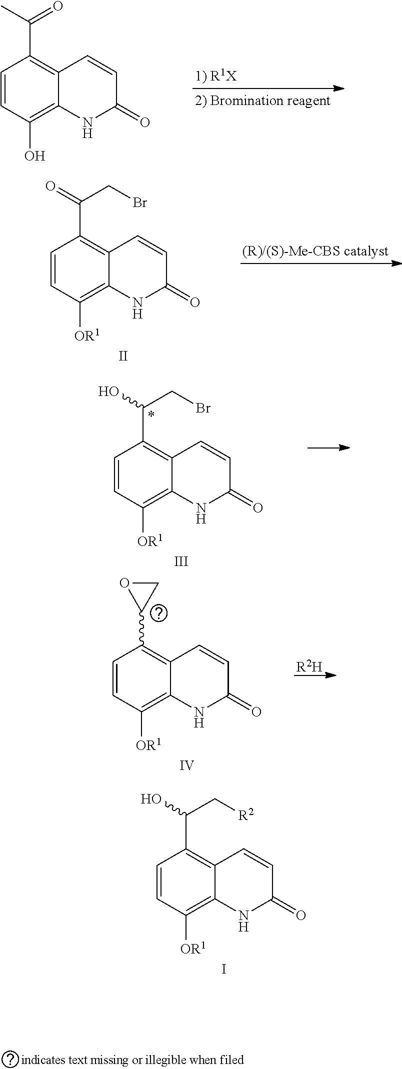

7. A method for preparing the compound according to claim 6, wherein,
in the reaction of compound II to prepare compound III, the solvent was selected from one or more of acetonitrile, dichloromethane, chloroform, tetrahydrofuran, N,N-dimethylformamide, dimethyl sulfoxide; The reaction temperature was −20° C. to 25° C.;
in the reaction for the preparation of compound IV from compound III: the solvent was chosen from one or more of acetonitrile, dichloromethane, chloroform, acetone, tetrahydrofuran, methanol, N,N-dimethylformamide, dimethyl sulfoxide or dioxane; The base is selected from potassium carbonate, sodium carbonate, sodium bicarbonate, sodium hydroxide, potassium hydroxide or triethylamine; The reaction temperature was 25° C. until heating reflux;
in the reaction to prepare compound I from compound IV, the solvent was selected from acetonitrile, N,N-dimethylformamide, dimethyl sulfoxide, n-butanol or diethylene glycol dimethyl ether; The reaction temperature was from 60° C. to 150° C.

8. A method for preparing the compound according to claim 7, wherein
in the reaction of preparation of compound III from compound II: anhydrous tetrahydrofuran was selected as solvent and the reaction temperature was −20° C. to 0° C.;
in the reaction of preparation of compound IV from compound III, acetone was chosen as the solvent, potassium carbonate or triethylamine was chosen as the base, and the reaction temperature was 55° C. to 65° C.;
in the reaction for the preparation of compound I from compound IV: n-butanol was chosen as the solvent and the reaction temperature was 110° C. to 120° C.

9. Application of the compounds according to claim 1 in the preparation of a drug for treatment of a disease associated with a malignant tumor.

10. Application of claim 9, wherein the disease associated with a malignant tumor is a tumor mediated by SRSF6 protein overexpression, comprising a colorectal tumor.

11. Application of the compounds according to claim 2 in the preparation of a drug for treatment of a disease associated with a malignant tumor.

12. Application of the compounds according to claim 3 in the preparation of a drug for treatment of a disease associated with a malignant tumor.

13. Application of the compounds according to claim 4 in the preparation of a drug for treatment of a disease associated with a malignant tumor.

14. Application of the compounds according to claim 5 in the preparation of a drug for treatment of a disease associated with a malignant tumor.
